(19) United States
(12) Patent Application Publication
(10) Pub. No.: US 2007/0203472 A1
(43) Pub. Date: Aug. 30, 2007

(54) Multi purpose probe

(76) Inventor: Ronen Nachmani, Moshav Gan Hadarom (IL)

Correspondence Address: PEARL COHEN ZEDEK LATZER, LLP, 1500 BROADWAY 12TH FLOOR, NEW YORK, NY, 10036, US

(21) Appl. No.: 11/654,689

(22) Filed: Jan. 18, 2007

(30) Foreign Application Priority Data
Jan. 19, 2006 (IL) 173251

(51) Int. Cl. A61M 25/00

(52) U.S. Cl. 604/523

(57) ABSTRACT

Probing method and apparatus for exploring and operating within a selected space. The apparatus comprises: an anchoring element and an everted tube. When the tube is moved in the selected space, the inner rim of the tube moves, while the outer rim, which is fixed to said anchoring point, remains static. The apparatus is also provided with at least one of a plurality of tools, inserted into at least a portion of the tube, for using within the selected space.

### CROSS REFERENCE TO RELATED APPLICATIONS

This application claims priority from Israeli Patent Application Number 173251, filed on Jan. 19, 2006, which is incorporated in its entirety herein by reference.

### FIELD OF THE INVENTION

The present invention relates to new apparatus and method for probing and operating within variety of inaccessible cavities and lumens.

### BACKGROUND OF THE INVENTION

Many tasks in everyday life require scanning and operating within a concealed inaccessible space. A variety of applications address this challenge. For example, in medical applications, different devices are inserted into the human body through small openings, usually using a catheter. These devices (for example endoscopes) are used for probing and operating internally, thus eliminating the need for surgery. Other probe applications include construction work, rescue missions, geological surveys, archeological excavations, and in many other tasks in which exploring cavities and conduits is needed.

Probing inaccessible areas involves many problems, some general in nature, and some specific for a given application. In medical applications, one of the most significant problems is the insertion of a probe and tools without causing damage to live tissue. The forces exerted during the insertion of a probe or tools may lead to blood vessel avulsion and trauma to tissue and internal organs. Thus a method of a traumatic blunt dissection and insertion is desired. Preferably a method, which includes an option for visual control during this process is desired. Several solutions address these problems.

For example, a catheter and an inverted balloon are disclosed in U.S. Pat. No. 4,271,839. This patent discloses a dilation catheter method and apparatus, which use an inverted balloon to reach and dilate a partially occluded section of a blood vessel. The balloon responds to the exertion of internal fluid pressure on the balloon. The catheter is designed to extrude through the occluded vessel in advance of substantial lateral expansion and, upon full extension, is adapted to expand to a condition at least partially dilating the occlusion. A cord connected to the balloon and extending through the catheter provides means whereby tension may be applied to the balloon to reinvert it within the catheter. A reservoir in communication with the inner lumen of the catheter provides a closed fluid-filled system whereby the balloon may be sequentially everted and reinverted without venting outside of the system. Single lumen everting balloon catheters, such as disclosed in this patent, do not provide an additional channel, which can be used for visual control tool such as an endoscope. If an endoscope were inserted into the disclosed single balloon lumen, the endoscope would be unable to track down to the distal part of the catheter due to the presence of the balloon positioned at the end of the catheter.

Double lumen everting balloon catheters, are disclosed in U.S. Pat. Nos. 4,479,497, 4,526,275 and 4,863,440. U.S. Pat. No. 4,863,440 further discloses the use of a through-lumen that permits the passage of an endoscope.

In U.S. Pat. No. 5,613,947, “Everting cannula apparatus and method”, a double-lumen balloon cannula and method were disclosed, for performing blunt dissection of an elongated non-spherical cavity under visual control. In this example, a first tubular lumen supports an endoscope, and a second lumen supports an inverted transparent balloon that is capable of being everted in response to fluid under pressure that is applied to it. The endoscope is selectively advanced within the everting balloon to visualize tissue in the elongated cavity through the balloon. In the disclosed configuration, the balloon does not interfere with the endoscope's view, thus the procedure may take place under visual supervision.

The principal of using an everted tube and pressure to mechanically operate it in an inaccessible zone is also demonstrated in U.S. Pat. No. 4,329,995. In this example an everted tube is used to seal and open the end of a catheter, serving as a pressure activated valve. More specifically, a catheter is passed through the nasopharynx into the trachea without contamination of the catheter lumen for obtaining uncontaminated sputum specimens. The catheter lumen is sealed to prevent entry of contaminants by the provision of a length of flexible, expandable tube placed over and attached to the distal end of the catheter. The flexible tube is rolled-up over a portion of its length and inverted into the unrolled portion, with the unrolled portion forming a cuff encircling the rolled-up portion to hold the rolled portion. A syringe pump connected to the opposite end of the catheter introduces fluid under pressure into the lumen of the catheter to expand the cuff and urge the rolled-up portion of the tube from the cuff, and to unroll the tube, whereby the lumen of the catheter is unsealed. An inner catheter is inserted into the lumen of the outer catheter and advanced there through and out the end beyond the tube for performing aspiration.

U.S. Pat. No. 4,576,205 uses similar principals, of an everting balloon-tube element, which is inverted, using pressure, to provide a forward insertion motion, for adding tubular lining material to pipe-lines. In this patent a special tubular lining material is inserted into the pipe-line and allowed to advance within the pipe-line with or without the aid of a leading rope-like elongated element, while turning the tubular lining material inside out under fluid pressure thereby applying the tubular lining material onto the inner surface of the pipe-line.

GB 640223 is another example of using the everted tube for working in inaccessible spaces. In this application the everted tube is used for concrete casting in construction work.

The present invention is aimed at providing a new solution using an everted tube. Disclosed are an apparatus and a method for probing and operating within a variety of inaccessible spaces such as: caves, buildings and structures, ruins and body lumens.

### SUMMARY OF THE INVENTION

There is thus provided, in accordance with some preferred embodiment of the present invention, a probing apparatus for exploring and operating within a selected space, the apparatus comprising:

an anchoring element;

an everted tube, whereby when the tube is moved in said space, the inner rim of the tube moves, while the outer rim, which is fixed to said anchoring point, remains static;

at least one of a plurality of tools, inserted into at least a portion of the tube, for using within the space.

Furthermore, in accordance with some preferred embodiments of the present invention, said tube is advanced or retracted by a propulsion mechanism, which provides pressure that applies forces across a front of an everted section of the tube.

Furthermore, in accordance with some preferred embodiments of the present invention, said tube is advanced or retracted by pressure gradients in said selected space.

Furthermore, in accordance with some preferred embodiments of the present invention, said tube is long as required.

Furthermore, in accordance with some preferred embodiments of the present invention, said tube is made from a collapsible material.

Furthermore, in accordance with some preferred embodiments of the present invention, said tube is elastic.

Furthermore, in accordance with some preferred embodiments of the present invention, the propulsion mechanism is based on medium selected from air, liquid or pressured gas.

Furthermore, in accordance with some preferred embodiments of the present invention, the propulsion mechanism further includes a control.

Furthermore, in accordance with some preferred embodiments of the present invention, said control comprises a pump, a processing unit and a control unit.

Furthermore, in accordance with some preferred embodiments of the present invention, the apparatus is further provided with a spool mechanism.

Furthermore, in accordance with some preferred embodiments of the present invention, the spool mechanism comprises a spool over which a portion of the tube is winded.

Furthermore, in accordance with some preferred embodiments of the present invention, the spool is operated by a motor.

Furthermore, in accordance with some preferred embodiments of the present invention, a distal end of the tube is interfaced by an interface to an external member.

Furthermore, in accordance with some preferred embodiments of the present invention, the external member is an external tube.

Furthermore, in accordance with some preferred embodiments of the present invention, the spool mechanism comprises a controller.

Furthermore, in accordance with some preferred embodiments of the present invention, the spool mechanism further comprises a processing unit and a display.

Furthermore, in accordance with some preferred embodiments of the present invention, said at least one of a plurality of tools is inserted within the tube.

Furthermore, in accordance with some preferred embodiments of the present invention, said at least one of a plurality of tools is inserted within the everted section of the tube.

Furthermore, in accordance with some preferred embodiments of the present invention, a conduit is defined within the tube for said at least one of a plurality of tools to be inserted in.

Furthermore, in accordance with some preferred embodiments of the present invention, said at least one of a plurality of tools comprises a medical tool.

Furthermore, in accordance with some preferred embodiments of the present invention, said at least one of a plurality of tools comprises a viewing tool.

Furthermore, in accordance with some preferred embodiments of the present invention, said forces across said front of said everted tube are used as a piston in said selected space.

Furthermore, in accordance with some preferred embodiments of the present invention, said front of said everted tube has a ring shape.

Furthermore, in accordance with some preferred embodiments of the present invention, there is provided a probing method for exploring and operating within a selected space, said method comprising:

everting a tube, thus creating an inner and outer rim of said tube;

fixing the outer rim of the tube to an anchoring point;

creating motion of said tube in said selected space, whereby during said motion, the inner rim of the tube moves, while said outer rim remains static;

inserting at least one set of tools into said tube.

Furthermore, in accordance with some preferred embodiments of the present invention, said motion of said tube includes advancing or retracting the tube by a propulsion mechanism, which provides pressure that applies forces across a front of an everted section of the tube.

Furthermore, in accordance with some preferred embodiments of the present invention, said motion of said tube includes advancing or retracting the tube by pressure gradients in said selected space.

Furthermore, in accordance with some preferred embodiments of the present invention, the propulsion mechanism is based on medium selected from air, liquid or pressured gas.

Furthermore, in accordance with some preferred embodiments of the present invention, said tube is wound or unwounded from a spool during the motion of said tube in said selected space, whereby any tube length required can be supplied.

Furthermore, in accordance with some preferred embodiments of the present invention, said at least one of a plurality of tools is inserted within the tube.

Furthermore, in accordance with some preferred embodiments of the present invention, said at least one of a plurality of tools is inserted within the everted section of the tube.

Furthermore, in accordance with some preferred embodiments of the present invention, at least one of a plurality of tools is inserted using a conduit within the tube.

Furthermore, in accordance with some preferred embodiments of the present invention, said at least one of a plurality of tools is able to perform medical operations.

Furthermore, in accordance with some preferred embodiments of the present invention, said at least one of a plurality of tools is able to provide viewing of said selected space.

Furthermore, in accordance with some preferred embodiments of the present invention, said forces across said front of said everted section of the tube, create a piston motion in said space.

Furthermore, in accordance with some preferred embodiments of the present invention, the motion of said tube is improved by using a tube with a ring shaped cross section.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 illustrates a schematic sectional view of a probing device in accordance with a preferred embodiment of the present invention.

FIG. 2 illustrates a spool mechanism device and control of that device, the spool being used for carrying the tube of the apparatus shown in FIG. 1 that is wound around it.

FIG. 3a illustrates a cross section of a collapsible material tube used in a probing device in accordance with a preferred embodiment of the present invention.

FIG. 3b illustrates a cross section of a different collapsible material tube used in a probing device in accordance with a preferred embodiment of the present invention.

FIG. 4a illustrates an overview of a collapsible everted tube, in accordance with a preferred embodiment of the present invention.

FIG. 4b illustrates the movement of a collapsible everted tube, in accordance with a preferred embodiment of the present invention.

### DETAILED DESCRIPTION OF PREFERRED EMBODIMENTS

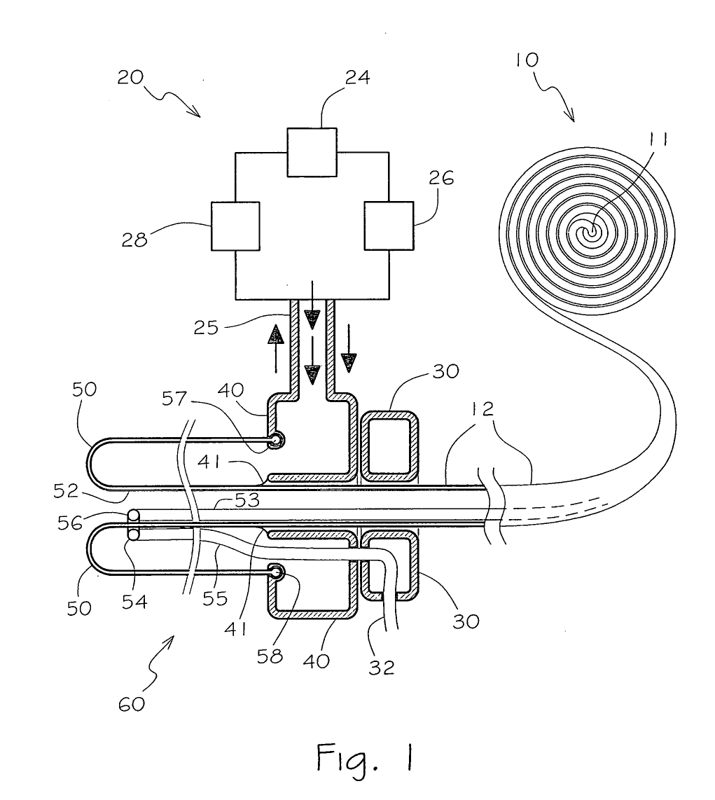

The present invention introduces a new multi-purpose probing method and system for exploring and operating with minimal friction to the surroundings within a variety of spaces. The invention is based on the use of a long everted tube, made from collapsible material (not necessarily flexible i.e. able to stretch), which when subjected to pressure, moves forward or backwards (FIG. 1) within a selected space.

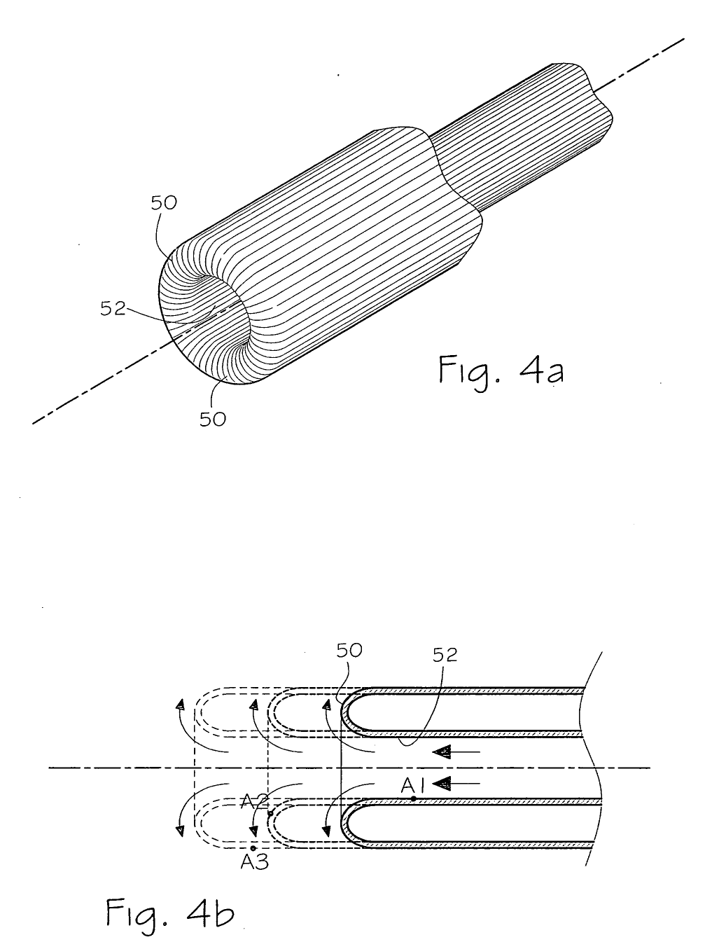

FIG. 4 illustrates a preferred embodiment of an everted tube, and its typical movement. A long as required collapsible tube is everted along its long axis, thus creating an inner rim (FIG. 4, 52) and an outer rim (FIG. 4, 50). In a preferred embodiment, the everted tube has a ring shaped cross section, as illustrated in FIG. 4a. However this is merely an example and other shapes are possible as well. In response to pressure applied, for example, between the outer (50) and inner (52) rims, the inner rim moves, while the outer rim (50), which is anchored to some point, remains static. This movement is illustrated in FIG. 4b by a series of two dashed lines, representing the new location of the everted tube front, as it moves forward (FIG. 4b). To further demonstrate a typical mode of motion of the everted tube, the location of a single point, A, on the inner rim of the tube, is traced in FIG. 4b. As pressure is applied to the tube front point A moves from location A1 to A2 and to A3 (FIG. 4b). This type of movement is characterized by minimal friction with its surrounding, since the outer rim of the (FIGS. 1 and 4, element 50) of the tube, which is in contact with the surrounding, is static, while only the inner rim (FIGS. 1 and 4, element 52) actually moves. In contrast to most inserted catheters used today, the forces, exerted during the insertion process of the everted tube, do not change as the length of the inserted tube increases. Thus the movement of the everted tube, makes it possible to avoid injuries or damage to the surrounding area, while probing or operating within a selected space.

The inserted tube can be used to insert additional tubes, which guide probes or tools into the selected space. Accordingly, the additional tubes, the probes and the tools are provided with a path, which does not include any lateral contact with the selected space. The creation of such a path is especially important in medical applications, where the surrounding tissue may be injured when tools are pushed back and forth in a standard method. Such a frictionless path is also significant in other probing applications, which are used in construction, caves, outer space, rescue and other probing tasks.

A preferred embodiment of the present invention is disclosed in FIG. 1. A long tube made from a collapsible material is used to aid in the insertion of probes and/or tools into a selected space. More specifically, FIG. 1 illustrates an apparatus, which includes: a spool mechanism (10), which is used to unwind and provide the required length of tube (12) to be inserted into a selected space; a pressure based propulsion system (20); and an everted tube front (60), which leads the tube insertion. The spool mechanism (10) provides the required length of tube (12), which is inserted into the selected space. A socket (11), which is part of the spool, helps hold the distal end of the tube on the spool (10). The other end of the tube (12), the free end, is guided by ring (30) towards ring (40). The tube free-end is everted to create a front, which includes an inner rim (52) and outer rim (50). The outer rim (50) is fixed to a special anchoring ring (40) at lip 57, to hold it firmly and prevent it from moving. In a preferred embodiment, ring (30) can include a mechanical propulsion mechanism to aid in inserting the tube (12) in to the selected space (for example motorized wheels).

In a preferred embodiment of the present invention, a pressure based propulsion system (20) is used to slide the tube (12) into the selected space. The propulsion system is based on air, liquid or pressured gas. The pressure is exerted via pipe (25) through ring (40) into the tube front between the outer rim (50) and the inner rim (52). The pressure, exerted at this point, creates an active front, which moves forward or backwards (if the pressure is inverted) in a sliding motion, since the outer rim (50) is fixed to ring (40), and the inner rim (52) is free to advance. If the tube front is placed at the entrance of a selected space, the tube (12) will slide into the selected space in a sliding motion. Thus as long as pressure is exerted on the tube front (between points 50, 52), the tube will keep on sliding with only minimal friction with its surroundings. To prevent the pressure from leaking, between the tube (12) and ring (40), a valve (41) is added. In a preferred embodiment, the pressure propulsion system includes a pump (24), a processing unit (26) for determining the best working parameters (or for predetermined parameters) and a control unit (28). The pressure from system (20) can be exerted in both directions—towards ring (40) and from ring (40), thus providing both forward and backward motion to the tube.

In other preferred embodiments the everted tube is advanced by using pressure gradients within the explored space.

The inserted tube (12) is used to guide exploring and operating tools, which are desired for operating in the selected space, without creating friction between them and the selected space. There are several options of inserting tools in to the selected space, while taking advantage of the path created by the everted tube (12) insertion. In one preferred embodiment, tools are inserted through pipe (25) in to the cavity created between tube walls 50 and 52—in to the tube front. The tools have no contact with their surroundings, thus no friction is created between them and the explored environment. Their only contact is with the inserted tube (12).

Alternatively or additionally, tools (56) are inserted through an additional tube (53), which is inserted inside everted tube (12). In this embodiment the additional tube (53) and tools (56) are winded inside tube (12) on spool (10). Again, in this embodiment, the inserted tools have no contact with the explored space. Their only contact is with tube (12). Thus damage to the surrounding is prevented or greatly minimized.

Alternatively or additionally, tools (54) are inserted through an additional tube (55), which is inserted through rings (30) and (40) in between rims (50) and (52) of tube (12), using a perpendicular tube extension (32). As before, the tools have no friction with the surrounding, since they are hosted within the inserted tube (12).

In another preferred embodiment several tool-sets are inserted through any combination of additional tubes, as disclosed above, (25, 53, and 55). An important aspect of the invention is that the additional tubes and tools, which reside inside the everted tube, are able to move in and out of the selected space, without any friction with the surrounding environment. They have contact only with the everted tube (12). Thus, the insertion of tools, as described, does not cause damage to the surrounding space. Examples of tools, which can be inserted through the described tubes, are: endoscopes, catheters, optical fibers, dissection knives and tweezers. These tools are merely examples of optional tools, used mainly in medical applications, and in no way restrict the scope of the invention.

In another preferred embodiment, the everted tube's ring shaped front is adapted to exert forward pressure, when moving forward and or inflating laterally, thus acting like some kind of a piston. It may also be used to create other types of work in probed cavities, such as hydraulic explosion, stone cutting or cracking. It may also be used to deliver TNT or other material to be used within the probed cavity.

An important aspect of the disclosed invention is that a “safe path”, at any length desired, for any kind of tools needed, is formed by the everted tube, and enables numerous options for safe exploring and operating in the selected space.

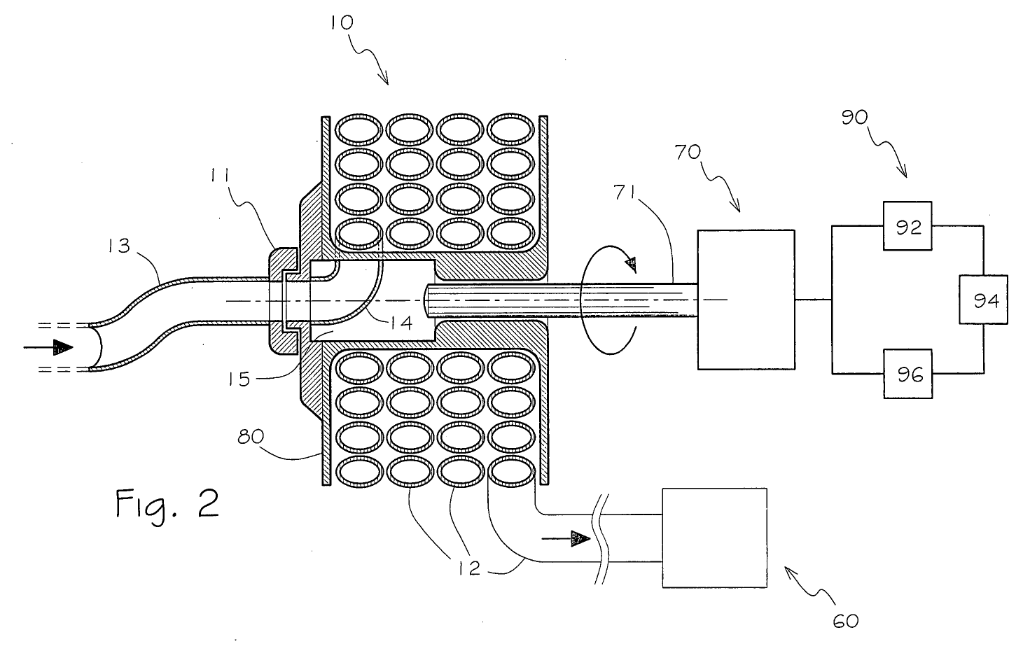

FIG. 2 discloses detailed sectional view of the spool mechanism (10), which enables providing the desired length of tube (12), at a controlled pace. More specifically, a spool (80) is used to wind tube (12). In a preferred embodiment, where the tools (56) are inserted through an additional tube (53), which resides within tube (12), the additional tube and tools are winded with tube (12) on the spool (80). The spool (80) is connected to a pivot (71), which is rotated by a propulsion unit (70). A control unit (90), which optionally includes a processing unit (92), a controller (94) and display (96), is responsible for monitoring the rate of unwinding. In a preferred embodiment, tube (12) is connected to rigid tube (14), which passes through block (15), and connects to external separate tube (13), through an intermediary socket (11), and provides an opening for everted tube (12), and for additional tube (53), which enables using inserts (56), within the tube system (12). In another preferred embodiment socket (11) also provides as interface to other systems, using tube (13).

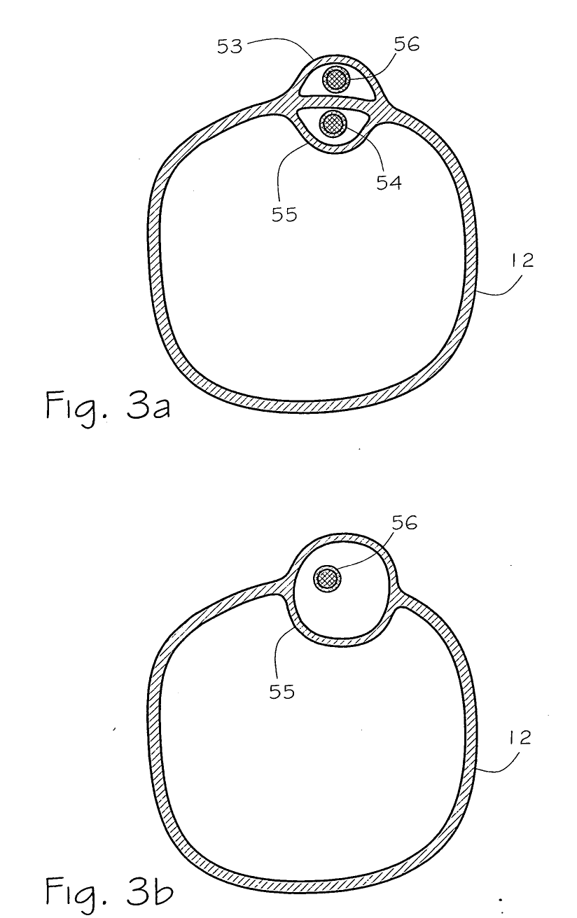

A cross section of everted tube (12) is disclosed in FIGS. 3a and 3b. FIG. 3a, demonstrates a preferred embodiment, which includes the insertion of two additional tubes (53 and 55), thus enabling insertion of two sets of insertion tools (56, 54). FIG. 3b demonstrates another preferred embodiment, where only one additional tube (55) is inserted with the everted tube (12), thus only a single set of tools (56) can be inserted with the everted tube (12).

The everted tube is made from a collapsible or pliable material that allows the tube to be everted. The particular choice of materials depends on the intended use and the anticipated surroundings. In a preferred embodiment the pliable material may be elastic as well.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the present invention.

What is claimed is:

1. A probing apparatus for exploring and operating within a selected space, the apparatus comprising:
an anchoring element;
an everted tube, whereby when the tube is moved in said space, the inner rim of the tube moves, while the outer rim, which is fixed to said anchoring point, remains static;
at least one of a plurality of tools, inserted into at least a portion of the tube, for using within the space.

2. The apparatus of claim 1, wherein said tube is made from a collapsible material.

3. The apparatus of claim 2, wherein said tube is elastic.

4. The apparatus of claim 1, wherein said tube is provided with a propulsion mechanism for advancing or retracting the tube, which provides pressure that applies forces across a front of an everted section of the tube.

5. The apparatus of claim 5, wherein the propulsion mechanism further includes a control.

6. The apparatus of claim 5, wherein said control comprises a pump, a processing unit and a control unit.

7. The apparatus of claim 1, further provided with a spool mechanism.

8. The apparatus of claim 7, wherein the spool mechanism comprises a spool over which a portion of the tube is winded.

9. The apparatus of claim 8, provided with a motor for operating the spool.

10. The apparatus of claim 8, wherein a distal end of the tube is equipped with an interface to an external member.

11. The apparatus of claim 10, wherein the external member is an external tube.

12. The apparatus of claim 7, wherein the spool mechanism comprises a controller.

13. The apparatus of claim 7, wherein the spool mechanism further comprises a processing unit and a display.

14. The apparatus of claim 1, wherein said at least one of a plurality of tools is inserted within the tube.

15. The apparatus of claim 1, wherein said at least one of a plurality of tools is inserted within an everted section of the tube.

16. The apparatus of claim 1, wherein a conduit is defined within the tube for said at least one of a plurality of tools to be inserted in.

17. The apparatus of claim 1, wherein said at least one of a plurality of tools comprises a medical tool.

18. The apparatus of claim 1, wherein said at least one of a plurality of tools comprises a viewing tool.

19. The apparatus of claim 1, wherein a front of said everted tube is shaped as a ring.

20. A probing method for exploring and operating within a selected space, said method comprising:
everting a tube, thus creating an inner and outer rim of said tube;
fixing the outer rim of the tube to an anchoring point;
creating motion of said tube in said selected space, whereby during said motion, the inner rim of the tube moves, while said outer rim remains static;
inserting at least one set of tools into said tube.

21. The method of claim 20, wherein said motion of said tube includes advancing or retracting the tube by a propulsion mechanism, which provides pressure that applies forces across a front of an everted section of the tube.

22. The method of claim 20, wherein said motion of said tube includes advancing or retracting the tube by pressure gradients in said selected space.

23. The method of claim 20, wherein said forces across said front of said everted section of the tube are used to create a piston motion in said space.
